(19) United States
(12) Patent Application Publication
(10) Pub. No.: US 2009/0099581 A1
(43) Pub. Date: Apr. 16, 2009

(54) METHODS AND APPARATUS FOR TREATING VASCULAR OCCLUSIONS

(76) Inventors: Daniel H. Kim, Houston, TX (US); Roy Chin, Pleasanton, CA (US)

Correspondence Address: PATTERSON & SHERIDAN, L.L.P., 3040 POST OAK BOULEVARD, SUITE 1500, HOUSTON, TX, 77056, US

(21) Appl. No.: 12/126,646

(22) Filed: May 23, 2008

Related U.S. Application Data
(60) Provisional application No. 60/939,890, filed on May 24, 2007.

(51) Int. Cl. A61B 17/22; A61M 29/00; A61B 17/32; A61B 19/00

(52) U.S. Cl. 606/159; 606/198; 606/167; 128/898

(57) ABSTRACT

Embodiments of the present invention are suitable for treating a vascular occlusion. In one embodiment, a tissue removal apparatus includes a catheter having a lumen; a rotatable shaft disposed in the lumen of the catheter; a tissue removal portion connected to the rotatable shaft; and an expandable element circumferentially disposed at a distal end of the catheter, wherein upon expansion, at least a portion of the expandable element extends past the distal end of the catheter. In another embodiment, the rotatable shaft may include an auger portion to facilitate withdrawal of the removed tissue. In yet another embodiment, the tissue removal portion may include a plurality of expandable cutting members.

### CROSS-REFERENCE TO RELATED APPLICATIONS

This application claims benefit of U.S. Provisional Patent Application Ser. No. 60/939,890, filed May 24, 2007, which application is incorporated herein by reference in its entirety.

### BACKGROUND OF THE INVENTION

1. Field of the Invention

The present invention generally relates to medical devices and methods for accessing a particular targeted area of a patient and removing tissue therefrom. More particularly, the present invention relates to medical devices and methods for treating vascular occlusions.

2. Description of the Related Art

The medical industry is constantly evolving through the adaptation of improved pharmaceutical, biotechnology, and medical device products and procedures. Techniques and technologies are being developed to treat internal areas of the body through less invasive means.

It is often desirable and frequently necessary to remove a portion of tissue from humans and other animals, particularly in the diagnosis and treatment of patients with arteriosclerosis, herniated disc or other spinal disorders, cancerous tumors, pre-malignant conditions, benign prostatic hyperplasia (BPH) or prostatic cancer, liver disease, breast disease including cancer, brain disease including cancer and other diseases or disorders at any location in a patient.

Arteriosclerosis is a common vascular disease in which a patient's blood vessels become hardened and blocked by plaque or clots that impede blood flow. If the restriction becomes severe, the vessel may totally occlude. Reduction in blood flow due to the restricted blood vessels may cause heart attack, stroke, or high blood pressure.

Several methods have been introduced to alleviate the effects of plaque build-up restricting the arterial lumen. One such method is a procedure termed angioplasty, which uses an inflatable device positioned in the artery to dilate the lumen at the stenosis. In general, a balloon is introduced into the artery in a deflated state and guided through the artery over a guide wire to a position adjacent the stenosis. Fluid from a fluid source is then infused into the balloon via the catheter to inflate the balloon. Inflation of the balloon dilates the lumen of the artery to increase blood flow therethrough. After dilation, the balloon is deflated and removed from the artery.

Another method for treating arteriosclerosis is known as atherectomy. Unlike angioplasty which only dilates the artery, atherectomy reduces the restriction in the lumen by removing the plague from the artery. An atherectomy procedure typically includes inserting a guide wire into the affected artery and advancing a cutting device over the wire until the cutting device is positioned adjacent the stenosis. The cutting device is then advanced into the stenosis to cut a channel through the plaque, thereby increasing blood flow through the artery.

Both of these methods use a guidewire to position the tool adjacent the stenosis. Use of the guidewire may also prevent the occurrence of a puncture or perforation by an atherectomy device. However, use of the guidewire requires the existence of a passage through the occlusion. Thus, in cases where the occlusion is too severe to be traversed by the guidewire, these methods may not be effective.

A further problem associated with the use of conventional devices is the risk of perforating the blood vessels. For example, a guidewire or cutting device, when advanced, may cause dissection of the tissues of the vessel wall instead of the occlusion, thereby creating a perforation in the vessels.

Another reason that conventional types of device are typically ineffective in treating total or near total occlusions is that conventional catheter shafts and guidewires do not perform well under the compressive loading and torque loading that are required in order to advance such conventional devices across a chronic total occlusion lesion.

There is a need, therefore, for apparatus and methods to treat arteriosclerosis without the use of a guidewire inserted through the occlusion. There is also a need for apparatus and methods for treating arteriosclerosis without perforating the artery or vessel.

### SUMMARY OF THE INVENTION

Embodiments of the present invention are suitable for treating a vascular occlusion. In one embodiment, a tissue removal apparatus includes a catheter having a lumen; a rotatable shaft disposed in the lumen of the catheter; a tissue removal portion connected to the rotatable shaft; and an expandable element circumferentially disposed at a distal end of the catheter, wherein upon expansion, at least a portion of the expandable element extends past the distal end of the catheter. In another embodiment, the rotatable shaft includes an auger portion. In yet another embodiment, the tissue removal portion comprises one or more blades. In yet another embodiment, the tissue removal portion includes a plurality of expandable cutting members. In yet another embodiment, a retractable conveying member is provided for expanding the plurality of expandable cutting members.

In yet another embodiment, the tissue removal apparatus includes a second expandable element. In yet another embodiment, the tissue removal apparatus includes a third expandable element. In yet another embodiment, the third expandable element is adapted to deliver a stent. In yet another embodiment, the tissue removal apparatus includes a vacuum source connected to the lumen of the catheter.

In yet another embodiment, a method of removing tissue from a blood vessel includes Inserting into the blood vessel a distal end of a tissue removing apparatus comprised of a catheter having a rotatable shaft disposed therein, a tissue removal portion connected to the rotatable shaft, and an expandable element circumferentially disposed at a distal end of the catheter; expanding the expandable element against the blood vessel; extending the tissue removal portion beyond the expandable element; rotating the tissue removal portion to remove the tissue, whereby the expandable element prevents the tissue removal portion from perforating the blood vessel.

In yet another embodiment, expanding the expandable element comprises expanding the expanding element such that a distal end of the expandable element extends beyond a distal end of the catheter. In yet another embodiment, the method includes withdrawal the removed tissue through the catheter. In yet another embodiment, the tissue removal portion comprises one or more blades. In yet another embodiment, the method includes expanding the one or more blades.

In yet another embodiment, a tissue removal apparatus for removing a tissue in a blood vessel includes a catheter having at least one lumen; a conveyor disposed in one of the at least one lumen of the catheter; a tissue removal portion connected to the shaft; and an expandable centralizer disposed at a distal end of the catheter, wherein upon expansion, the expandable centralizer is adapted to centralize the tissue removal portion in a blood vessel. In yet another embodiment, at least a portion of the expandable centralizer extends past the distal end of the catheter after expansion. In yet another embodiment, the shaft is rotatable.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a cross-sectional view of an occluded artery.

FIGS. 2A-2E are various views of an embodiment of a vascular surgical device. FIGS. 2A-2C show sequential activation of the surgical device.

FIGS. 3A-3F illustrate sequential operation of the surgical device of FIG. 2A to treat the occlusion.

FIGS. 4A-4D are various views of another embodiment of a vascular surgical device.

FIGS. 5A-5D are various views of yet another embodiment of a vascular surgical device.

FIGS. 6A-6G are various views of yet another embodiment of a vascular surgical device.

FIGS. 7A-7F are various views of yet another embodiment of a vascular surgical device.

FIGS. 8A-8B are various views of yet another embodiment of a vascular surgical device.

FIGS. 9A-9F illustrate sequential operation of the surgical device of FIG. 8A to treat a partially occluded artery.

FIGS. 10A-10B are various views of yet another embodiment of a vascular surgical device.

FIGS. 11A-11D illustrate sequential operation of the surgical device of FIG. 10A to position a stent in the artery.

### DETAILED DESCRIPTION

Embodiments of the present invention provide methods and apparatus for the treatment of vascular occlusions. In one embodiment, a vascular surgical device comprises a catheter having a lumen, a rotatable shaft disposed in the lumen of the catheter, a tissue removal portion connected to the rotatable shaft; and an expandable centralizer circumferentially disposed at a distal end of the catheter, wherein upon expansion, at least a portion of the expandable centralizer extends past the distal end of the catheter.

In one embodiment, the occlusion may be treated using a catheter having a distal tip fitted with a concentric balloon or other concentric expandable device. During insertion of the catheter into the blood vessel, the balloon is deflated sufficiently to allow movement with the catheter. Following delivery of the balloon to the occluding vascular lesion to be treated, the balloon is inflated such that the balloon becomes stabilized or anchored within the blood vessel to maintain the entire catheter shaft in the center lumen of blood vessel.

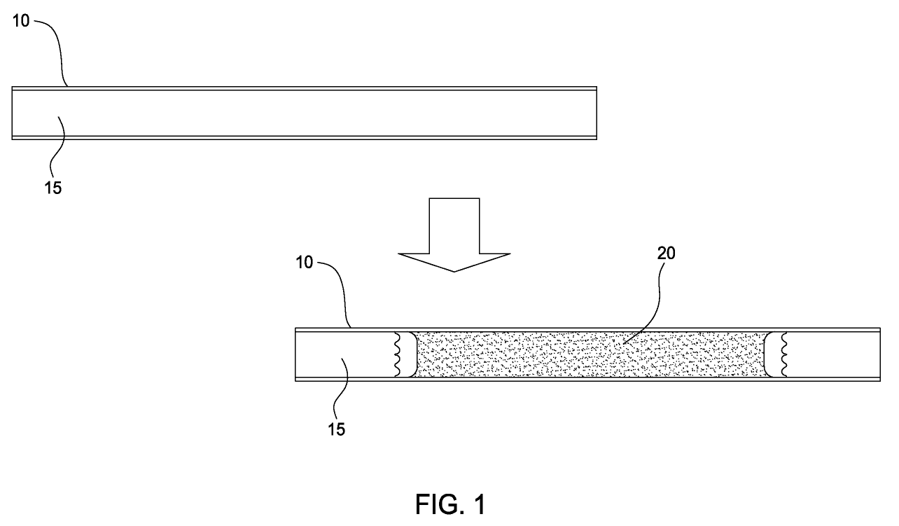

FIG. 1 shows a cross-sectional view of a diseased artery 10 suffering from a plaque 20 build up that occludes the lumen 15 of the artery 10. Although the artery 10 is shown as being totally occluded, embodiments of the present invention may be used to increase blood flow through a partially occluded artery or blood vessel.

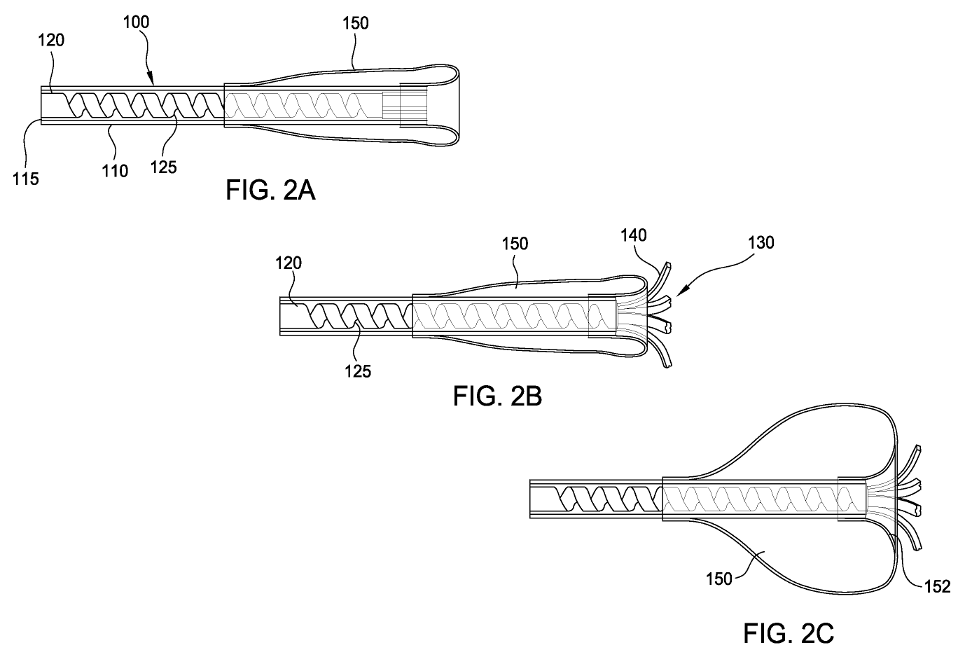

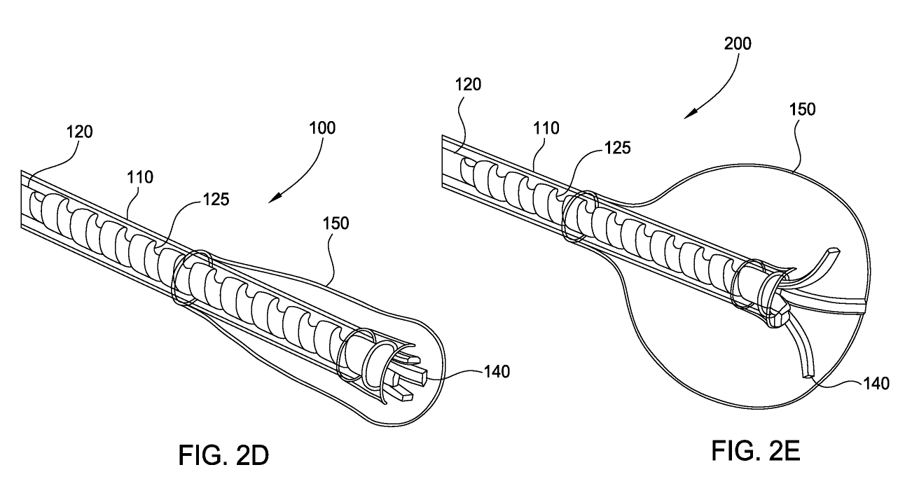

Embodiments of the present invention provide methods and apparatus for treating the occlusion to increase the blood flow through the artery 10. FIGS. 2A-2E are various views of an embodiment of a vascular surgical device 100 adapted to remove plague 20 or other tissue obstructing the artery 10. The surgical device 100 includes a catheter 110 having one or more lumens for transporting a tool, fluid, or tissue. Each lumen may be independently used to supply or withdraw the tool, fluid, or tissue as necessary. In one embodiment, the catheter 110 may have three or more lumens. As shown, a rotatable shaft 120 is disposed in a first lumen 115 of the catheter 110. The rotatable shaft 120 includes an auger portion 125 and a tissue removal member 130 disposed at its distal end. The auger portion 125 may be a spiral recess formed on the outer surface of the shaft 120. In addition to being rotatable, the rotatable shaft 120 is also axially movable in the catheter 110. In one embodiment, the shaft 120 has a length in a range between about 5 inches and 40 inches and an outer diameter in a range between about 1 mm to 10 mm.

In FIGS. 2A-2E, the tissue removal member 130 is shown as a plurality of expandable blades 140. FIG. 2A shows the blades 140 positioned adjacent the distal end of the catheter 110. As shown, the tissue removal member 130 has four blades 140, although any suitable number of blades may be used, for example, three or five blades. One end of each blade 140 is attached to the shaft 120 and the other end is a free end. See FIG. 2D. Suitable body shapes for the blades include a thin, flat strip body; a wire body; a triangular body; and any other suitable shape known to a person of ordinary skill. In one embodiment, the blades 140 may be made of metal and have an arcuate body length in its natural state. To actuate the tissue removal member 130, the blades 140 are extended out of the catheter 110 and rotated. FIGS. 2B, 2C, 2E show the blades 140 fully extended. The cutting radius, which determines the size of the channel formed through the occlusion, may be controlled by controlling the length of the blade 140 extending out of the catheter 110. As the blades 140 extend, the cutting radius increases due to the arcuate shape of the blades. Further, the rate of rotation of the blades 140 may also increase the size of the cutting radius. Rotation and advancement of the free end of the blade 140 removes plague in its path of rotation to form a channel through the occlusion. The blades 140 may be advanced until they cross the entire occlusion. The blades 140 may extend beyond the catheter 110 by a distance in a range of 0.1 inch to about 2 inches, preferably, about 0.20 inch to about 1.25 inches. Exemplary material for the blades includes nitinol, stainless steel, and other suitable metal as is known to a person of ordinary skill in the art.

To prevent perforation of the arterial wall, an expandable centralizer may be disposed on the catheter. In one embodiment, the centralizer is a forward looking balloon 150 disposed at the distal end of the catheter 110. The balloon 150 is circumferentially positioned around the catheter 110 such that the catheter 110 extends axially through the balloon 150. The balloon 150 may be a tear-drop shaped balloon 150 with the wider diameter portion closest to the distal end of the catheter 110. The radius of the widest portion should be larger than the cutting radius of the blades 140. It must be noted that the balloon may be any suitable shape known to a person of ordinary skill in the art, for example, spherical or cylindrical shapes. In one embodiment, the distal end of the balloon 150 extends past the distal end of the catheter 110 but is behind the free end of the extended blades 140. This “forward looking” position minimizes the possibility of arterial wall perforation by the blades 140. Also, the balloon 150 may act as a dilator to keep the artery expanded and the arterial wall away from the blades 140. In another embodiment, the balloon 150 may provide a funnel shaped cavity 152 connected to the lumen 111 of the catheter 110 to facilitate the withdrawal of the removed plague into the lumen 111, as illustrated in FIG. 2C. In another embodiment, the balloon 150 may provide an atraumatic surface, when not inflated, to advance the catheter without causing injury to the vessels wall.

The balloon 150 may be disposed on the catheter 110 using any suitable method known to a person of ordinary skill in the art. In one embodiment, both ends of the balloon 150 may be connected to the catheter 110 using adhesive, heat bond, welding, or other suitable method. To create the “forward looking” position, the distal end is folded back before connection to the catheter 110. The balloon 140 may be inflated using a fluid such as air, water, saline, contrast agent, and combinations thereof. The inflation fluid may be supplied through one or more lumens of the catheter 110. The balloon 150 may be manufactured from polyurethane or other suitable expandable material. The wall thickness of the balloon may be from about 0.001 inches to 0.018 inches, preferably from about 0.002 inches to 0.010 inches. The softness of the balloon material may be from about 20-80 durometers, preferably from about 35-60 durometers.

The rotatable shaft 120 may be connected to a control unit having a motor for rotating the shaft 120. The control unit may be equipped with a motor control to turn the motor on and off and a power source such as a battery. A vacuum source may be connected to the control unit for extracting tissue removed by the tissue removal member 130. The motor turns a central shaft 120 which spins the auger 125 and the blades 140. Movement of the auger 125 along with the suction supplied by the vacuum source aspirates the tissue out of the target area. An exemplary control unit is disclosed in U.S. Provisional Patent Application No. 60/891,177, filed on Feb. 22, 2007 by Kim et al., which application is herein incorporated by reference in its entirety.

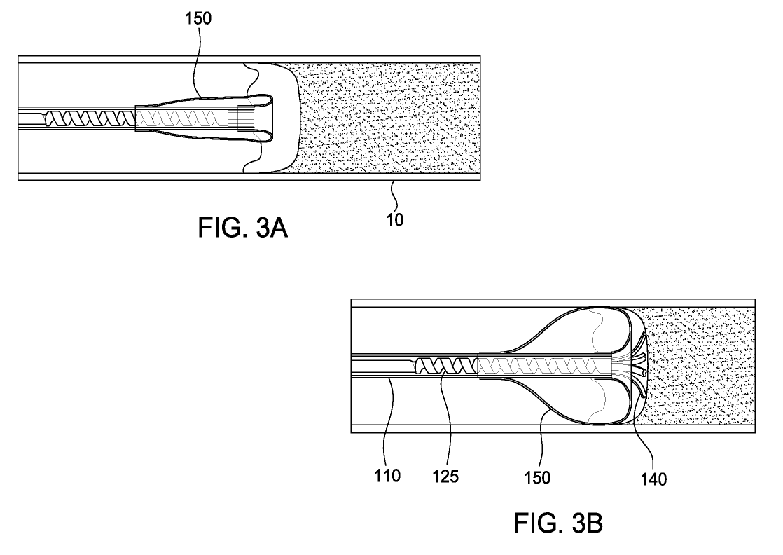

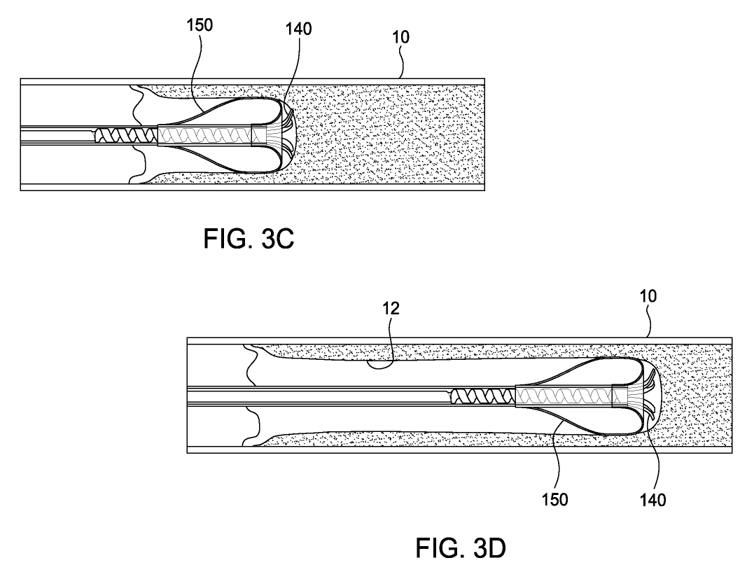

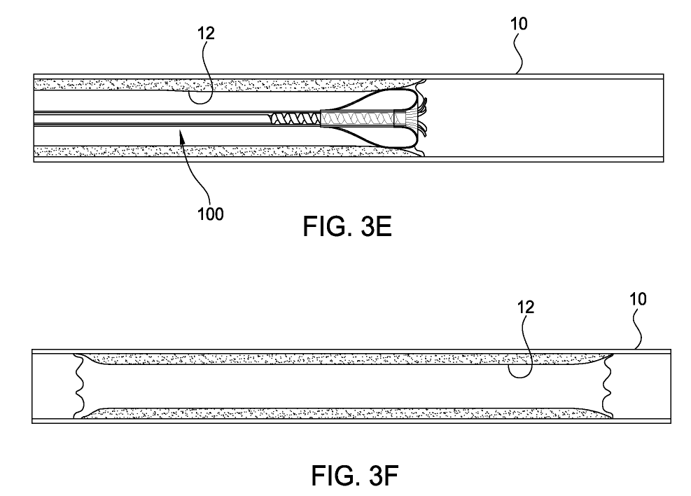

In operation, the surgical device 100 is inserted into the artery 10 and the blades 140 are positioned adjacent the occlusion. See FIG. 3A. The blades 140 are inserted in the unexpanded state. Thereafter, fluid is supplied through the second lumen to inflate the balloon 150. See FIG. 3B. After the blades 140 are extended, the motor is turned on to rotate the blades 140, and the blades 140 are advanced axially relative to the balloon 150 to remove the plague. The inflated balloon 150 prevents the blades 140 from perforating the artery 10 and also maintains the blades 140 in a substantially centralized position. The removed plague is drawn into the lumen 111 of the catheter 110 by the auger portion 125. The wider opening of the funnel shaped cavity 152 of the balloon 150 facilitates the capture of the removed plague into the lumen 111. Suction through the suction port may be used to increase the tissue withdrawal rate. After a short distance, the surgical device 100 must be repositioned closer to the unremoved plague. The surgical device 100 may be repositioned after cutting a length between about 1 mm to 1 cm, preferably, about 2 mm to 5 mm. To reposition the surgical device 100, the balloon 150 is deflated to allow movement of the balloon. At the new position, the balloon 150 is again inflated into contact with the artery and the blades 140 are rotated to remove the plague. See FIGS. 3C, 3D. The surgical device 100 is continuously repositioned to remove the plague until a channel 12 is formed through the occlusion. See FIGS. 3E, 3F. Because repositioning the surgical device 100 requires a repetitive deflation and inflation process, deflation of the balloon 150 for movement should be kept at a minimum amount required in order to increase the efficiency of the process.

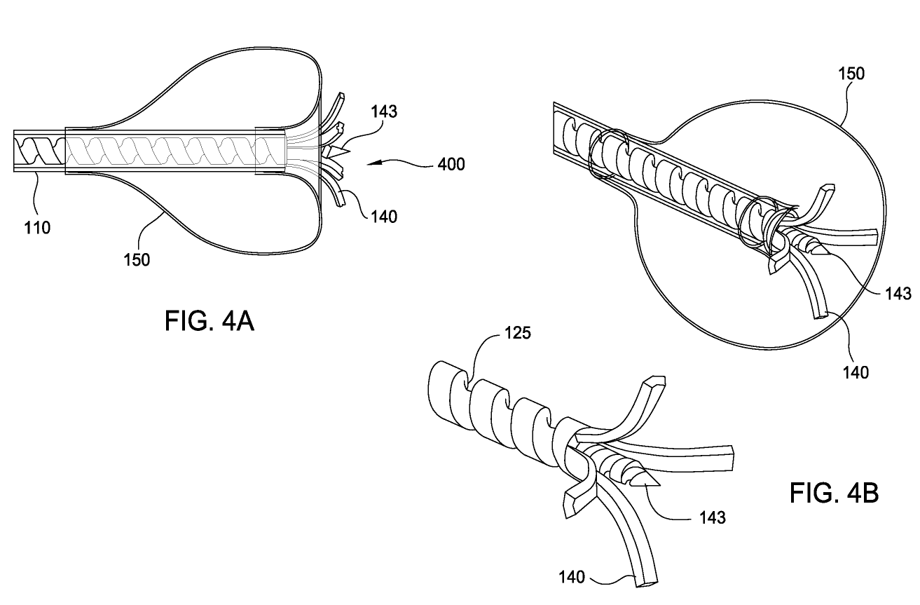

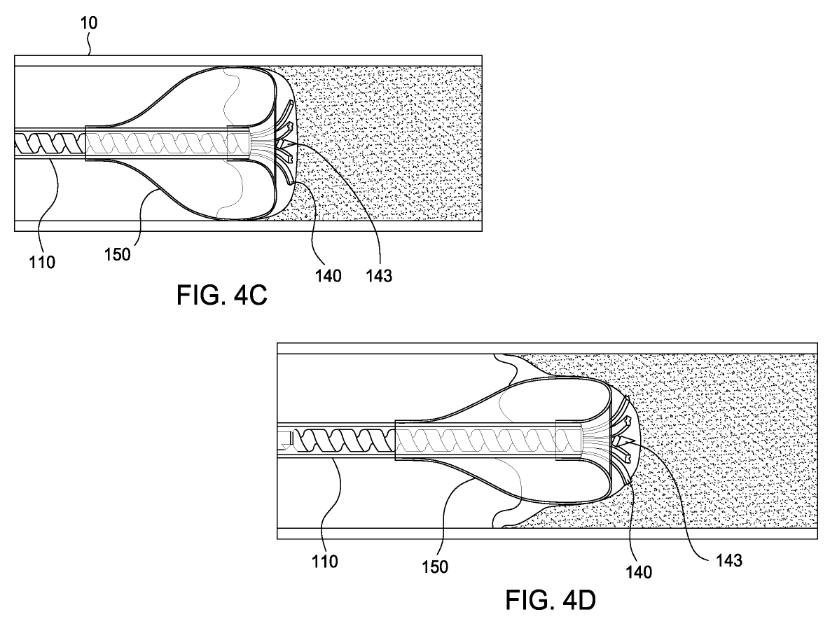

FIGS. 4A-4B illustrate another embodiment of a vascular surgical device 400. In this embodiment, the surgical device of FIG. 2 is modified to include a central cutting member 143. The central cutting member 143 is disposed in the middle of the blades 140. The central cutting member 143 provides additional tissue removing function in the central areas of the cutting radius. The cutting member 143 may include a sharp front end and an auger portion to facilitate removal of removed tissue. The central cutting member 143 may facilitate removal of the plague in the central portion of the cutting zone of the blades 140.

In operation, the surgical device 400 is inserted into the artery 10 and the blades 140 are positioned adjacent the occlusion. See FIG. 4C. As shown, the balloon 150 has been expanded against the arterial wall, and the blades 140 have been extended out of the catheter 110. Then, the motor is turned on to rotate the blades 140, and the blades 140 are advanced axially relative to the balloon 150 to remove the plague. The inflated balloon 150 prevents the blades 140 from perforating the artery 10. The removed plague is drawn into the catheter 110 by the auger portion. After a short distance, the balloon 150 is deflated and the surgical device 400 is repositioned closer to the unremoved plague. FIG. 4D shows surgical device 400 at a new position. After repositioning, the balloon 150 is again inflated into contact with the artery and the blades 140 are rotated to remove the plague. This process may be repeated until a channel 12 is formed through the occlusion.

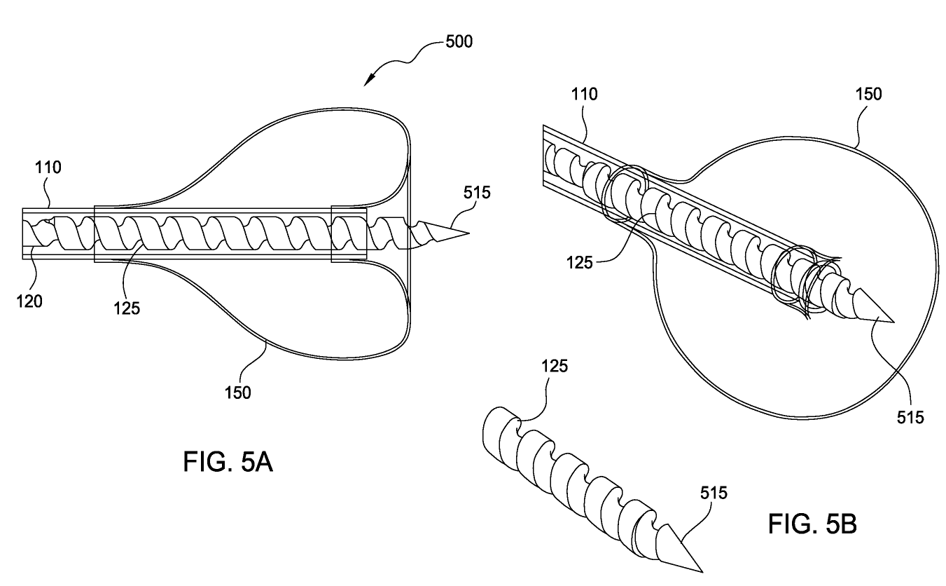

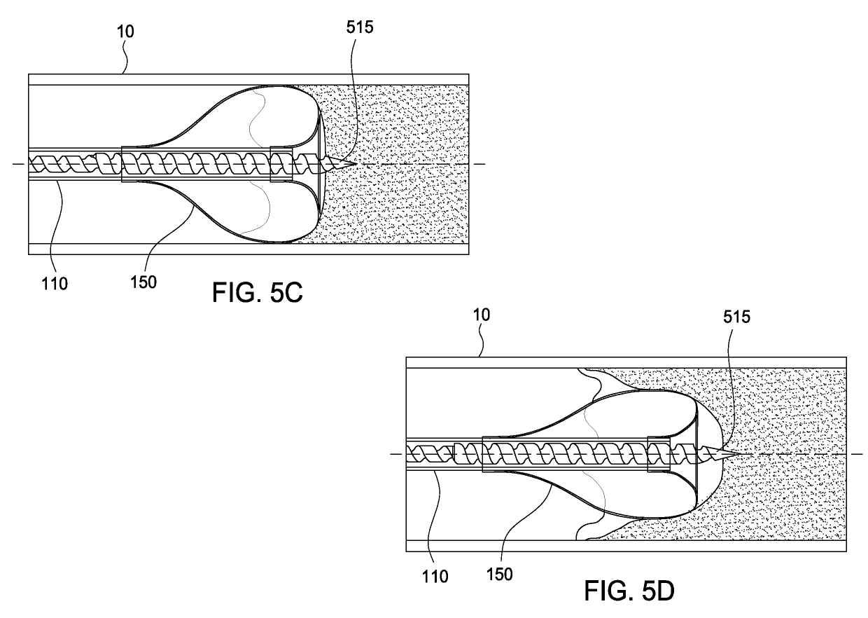

FIGS. 5A-5B illustrates another embodiment of a vascular surgical device 500. In this embodiment, the tissue removal member is a central cutting member 515. The central cutting member 515 may be integrated with the auger portion 125 of the shaft 120 such that the end of the auger portion 125 is provided with a sharp distal end which can penetrate the plague. This device 500 may be used to form a small passage through the occlusion such that a tool such as a guide wire may pass through the occlusion.

In operation, the surgical device 500 is inserted into the artery 10 and the central cutting member 515 is positioned adjacent the occlusion. See FIG. 5C. As shown, the balloon 150 has been expanded against the arterial wall, and the central cutting member 515 has been extended out of the catheter 110. Then, the central cutting member 515 is rotated and advanced axially relative to the balloon 150 to remove the plague and form a passage through the occlusion. The removed plague is drawn into the catheter 110 by the auger portion. After a short distance, the balloon 150 is deflated and the surgical device 500 is repositioned closer to the unremoved plague. FIG. 5D shows surgical device 400 at a new position. After repositioning, the balloon 150 is again inflated into contact with the artery and the the central cutting member 515 is rotated to remove the plague. This process may be repeated until the passage is formed through the occlusion.

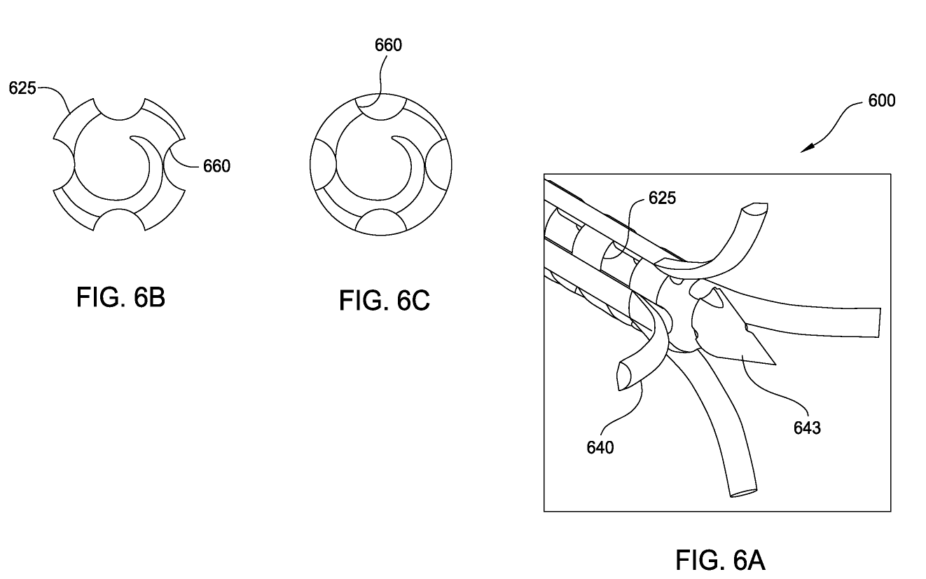

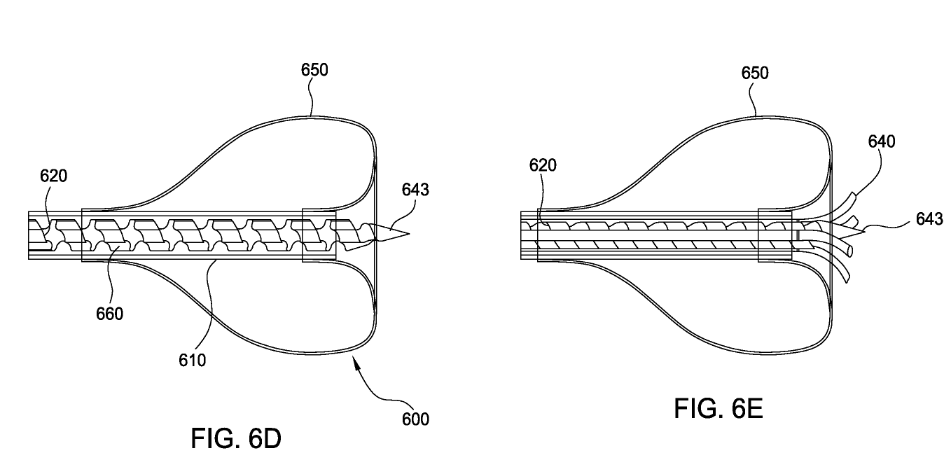

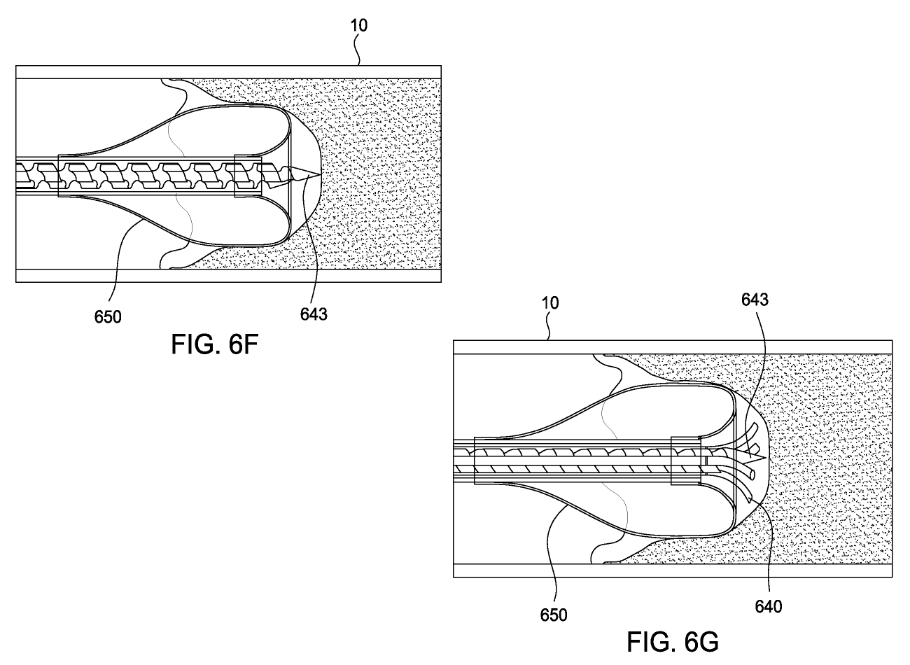

FIGS. 6A-6C illustrates another embodiment of a vascular surgical device 600. In this embodiment, the rotatable shaft includes an auger portion 625 and a sharp distal end 643. See FIG. 6A. The shaft also includes one or more recesses 660 that run longitudinally along the length of the shaft. See FIGS. 6B and 6C. FIG. 6B is front view of the distal end of the auger portion 625. As shown, four recesses 660 are circumferentially spaced around the outer surface of the shaft. The recesses 660 provide the flexibility of inserting and retrieving the blades 640, as necessary. In one embodiment, the recesses 660 are sized such that the outer surface of the blades 640 is substantially flushed with the outer surface of the shaft, as shown in FIGS. 6A, 6C. In this respect, the annular area between the shaft and the catheter is maximized for fluid flow and tissue removal.

FIG. 6D shows the surgical device 600 in operation. As shown, the balloon 650 is inflated and the distal end of the shaft 620 is extended out of the catheter 610. It can also be seen that the blades have not been inserted in the recesses 660. In this position, the shaft 620 may be rotated to form a pilot hole in the occlusion, as shown in FIG. 6F. In FIG. 6E, four blades 640 are inserted along the recesses 660 until the distal end of the blades 640 are past the balloon 650. It must be noted that the blades 640 may reside in the catheter while the shaft 620 is being rotated to form the pilot hole. In this respect, the blades 640 travel a shorter distance before extending out of the catheter 610. After insertion, the shaft 620 and the blades 640 are rotated to enlarge the pilot hole and form a passage through the occlusion, as shown in FIG. 6G.

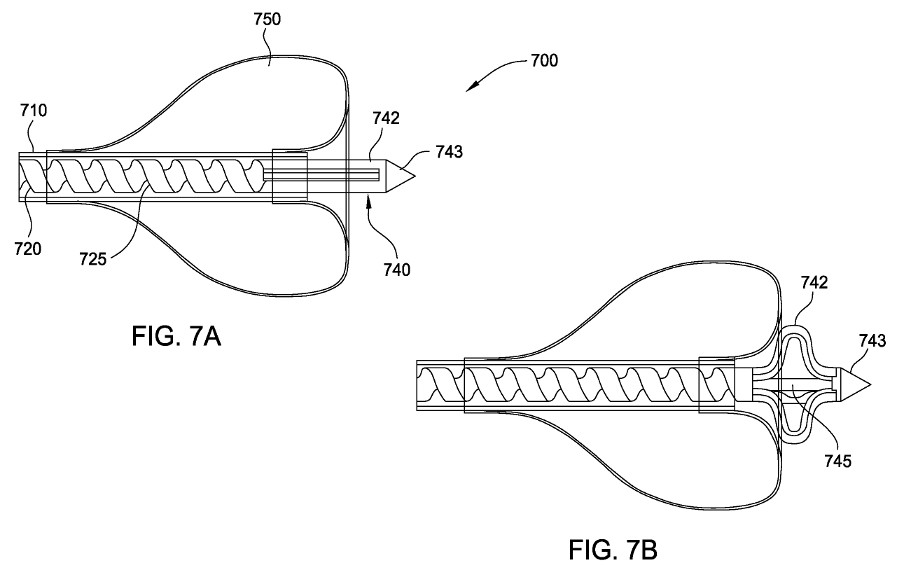

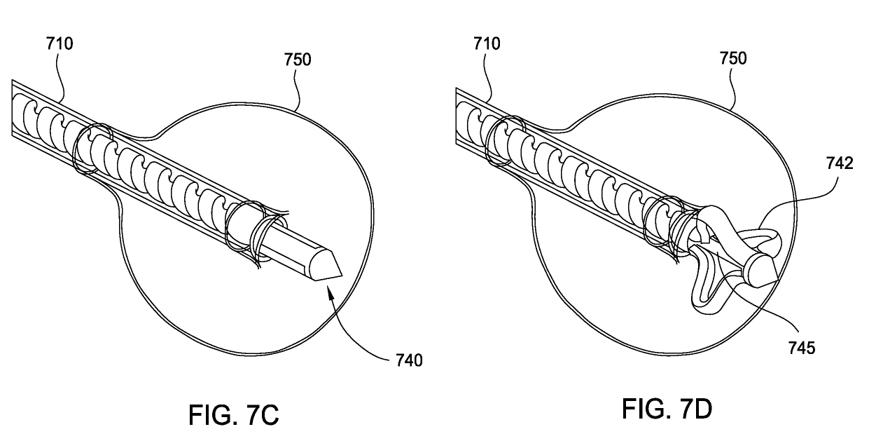

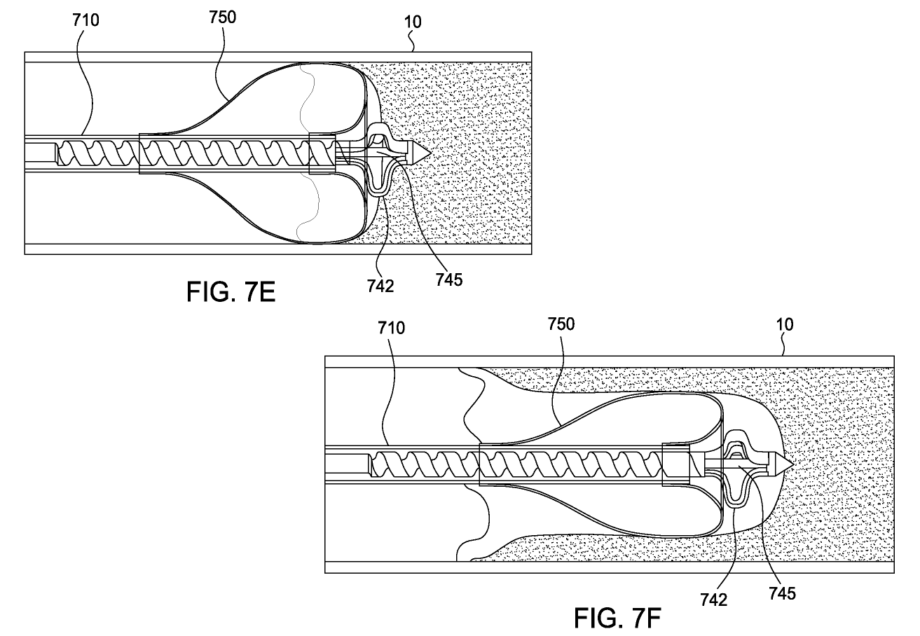

FIGS. 7A-7B illustrates another embodiment of a vascular surgical device 700. The device 700 includes a rotatable shaft 720 axially positioned in a catheter 710. The rotatable shaft 720 includes an auger portion 725 and a tissue removal member 740 at its distal end. The tissue removal member 740 includes four blades 742 attached to the shaft 720 at one end and a pointed tip 743 at another end. The blades 742 are made of a flexible metal. The blades 742 are positioned circumferentially around a wire 745, which has one end attached to the pointed tip 743. The wire 745 extends along a longitudinal channel through the shaft 720 to the control unit of the surgical device 700. In use, the shaft 720 is inserted into the artery with the blades 742 in the unexpanded position, as shown in FIG. 7A. After inflation of the balloon 750, the blades 742 may be expanded to increase its cutting radius. In this respect, the wire 745 is retracted relative to the blades 742 to pull the pointed tip 743 toward the catheter 710. The retraction causes the blades 742 to bow outward resulting in radial expansion of the blades 742, as shown in FIG. 7B. As more wire is retracted, the expansion increases. Thus, the diameter of the tissue removal member 740 may be controlled by controlling the extent of the wire 745 retraction. In another embodiment, a shaft, a cable, or other retractable conveying member may be used instead of a wire to extend or retract the blades.

In another embodiment, the surgical device 700 may be operated to enlarge the lumen as discussed. However, just before surgical device 700 breaks through the occlusion, the wire 745 is extended to bring the blades 742 back to its unexpanded state. Thereafter, the blades 743 are rotated to make a small opening to break through the occlusion. Thereafter, a second balloon disposed on a secondary catheter may be inserted through a third lumen in the catheter 710. The second balloon is delivered through the catheter 710 and the small opening to the other side of the occlusion. Then, the second balloon is inflated to seal off that side of the artery. The wire 143 is again retracted to expand the blades 742, and the blades 742 are rotated to form a larger opening through the occlusion. In this respect, the second balloon and the forward looking balloon 750 cooperate to trap the plague or debris for withdrawal through the catheter 710. In this manner, removed plague or debris are not allowed to flow downstream of the artery.

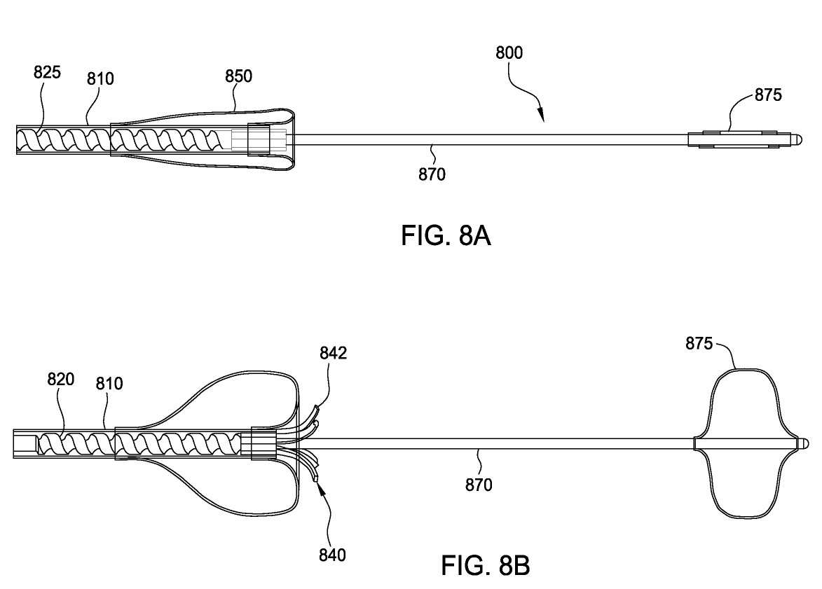

FIGS. 8A-B illustrate another embodiment of a vascular surgical device suitable for treating a partially occluded artery. FIG. 8A shows the surgical device in an inactivated state, and FIG. 8B shows the surgical device in an activated state. The surgical device 800 includes a rotatable shaft 820 axially positioned in a catheter 810. The rotatable shaft 820 includes an auger portion 825 and a tissue removal member 840 at its distal end. The tissue removal member 840 includes four blades 842 having one end attached to the shaft 820 and another end is a free end. The catheter 810 is equipped with a forward looking balloon 850 as described herein. A conveying tubular such as a second catheter 870 is inserted through a channel in the shaft 820 and between the four blades 842. The distal end of the second catheter 870 includes a second inflatable member such as a balloon 875 disposed circumferentially around the second catheter 870. The balloon 875 may be inflated with fluid supplied through a lumen of the second catheter 870. In another embodiment, the conveying tubular may be a flexible tubular rod.

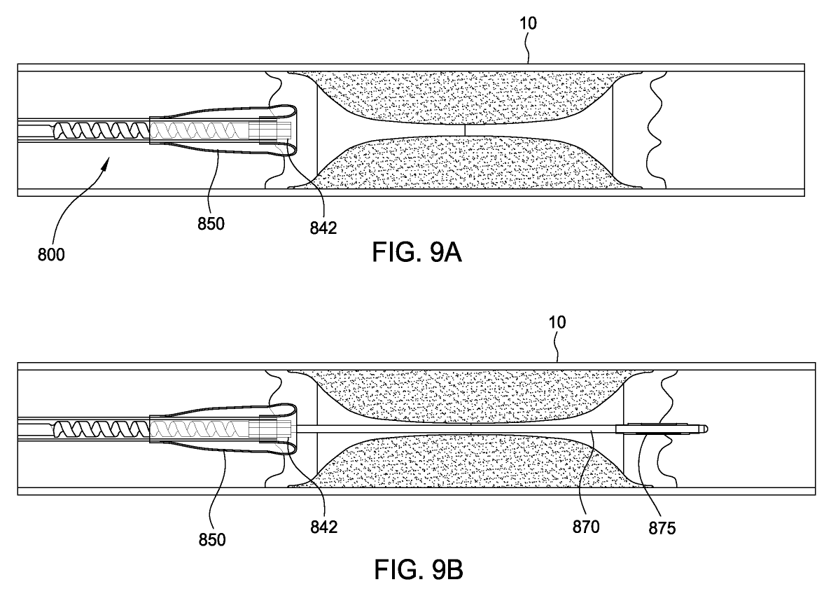

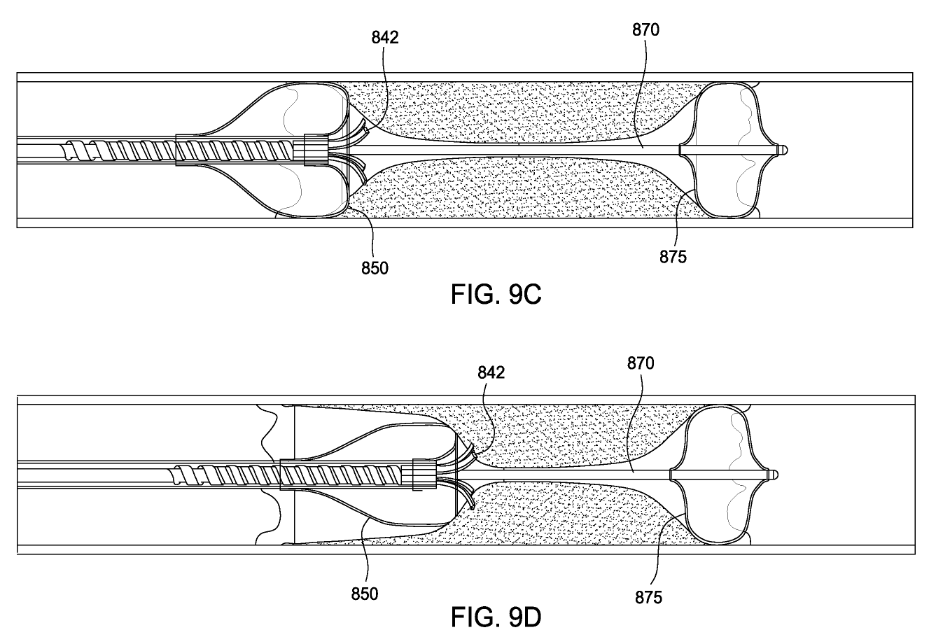

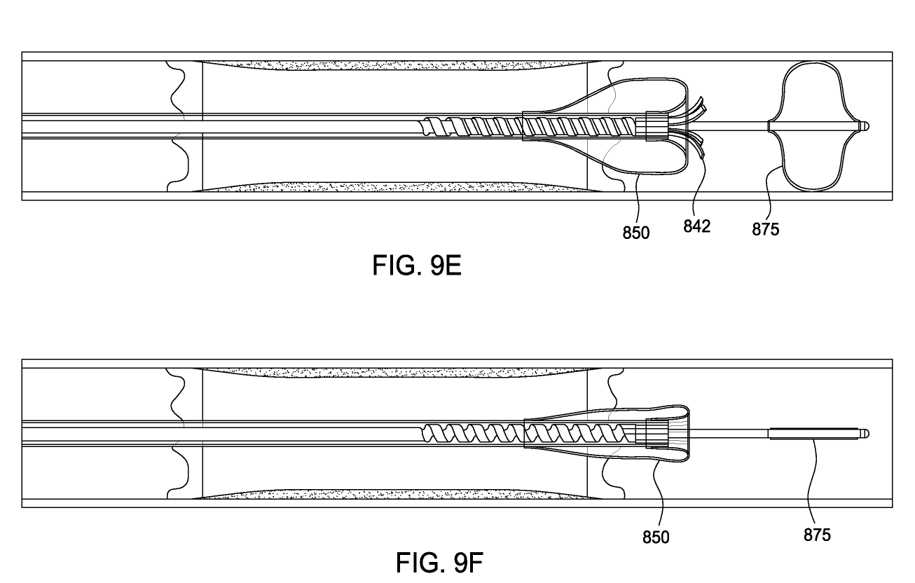

FIGS. 9A-F show the sequential operation of the surgical device 800. In FIG. 9A, the surgical device 800 is shown positioned adjacent a partially occluded artery. It can be seen that the blades 842 and the balloon 850 have not been expanded. Initially, the secondary catheter 870 is extended out of the surgical device 800 and advanced through the opening in the occlusion to position the second balloon 875 on the other side of the occlusion, as shown in FIG. 9B. In FIG. 9C, fluid is supplied to both balloons 850, 875 for expansion of the balloons 850, 875. The blades 842 are extended out and rotated to enlarge the lumen of the artery. The blades 842 are advanced until repositioning of the surgical device 800 is required. To reposition the surgical device 800, rotation of the blades 842 is stopped, and the forward looking balloon 850 is deflated. The blades 842 and the balloon 850 are then advanced toward the occlusion while the second balloon 875 remains in position. Then, the balloon 850 is inflated and the blades 842 are rotated to continue removing the occlusion, as shown in FIG. 9D. This removal process continues until the blades 842 reach the end of the occlusion. Just prior to break through, the second balloon 875 may be deflated and repositioned upstream to provide room for the blades 842 to advance, as shown in FIG. 9E. Then, the blades 842 may be rotated to break through the occlusion. Thereafter, the blades 842 are retracted and the balloons 850, 875 are deflated for removal from the patient, as shown in FIG. 9F.

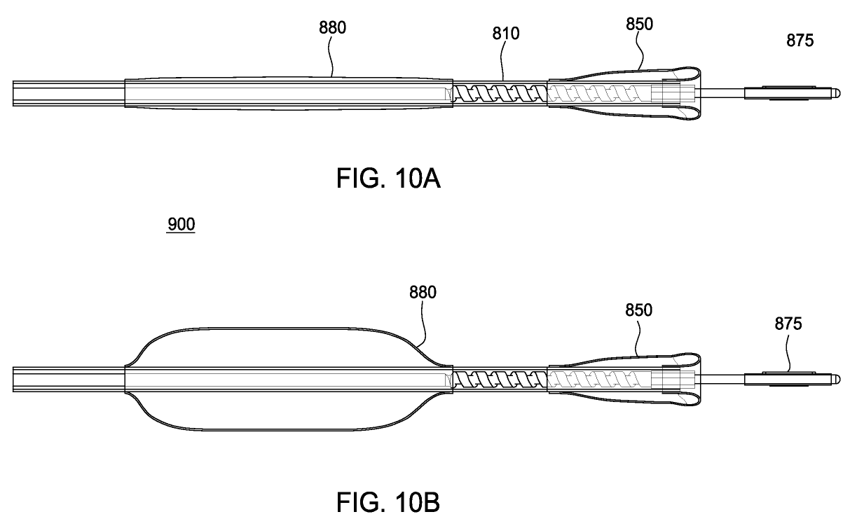

FIG. 10A shows another embodiment of a vascular surgical device 900. This surgical device 900 may be used to install a stent in the artery to maintain the enlarged lumen. The surgical device 900 is substantially similar to the surgical device 800 shown in FIGS. 8 and 9. As such, similar features are denoted with the same reference numbers to maintain clarity. The exception being that the surgical device 900 in FIG. 10 further includes an inflatable member such as a dilatory balloon 880 disposed around the catheter 810 downstream from the forward looking balloon 850. The dilatory balloon 880 may be inflated using fluid supplied through another lumen in the catheter 810. In this respect, each balloon 850, 875, 880 on the surgical device 900 may be independently inflated or deflated. FIG. 10B shows the dilatory balloon 880 in the inflated position.

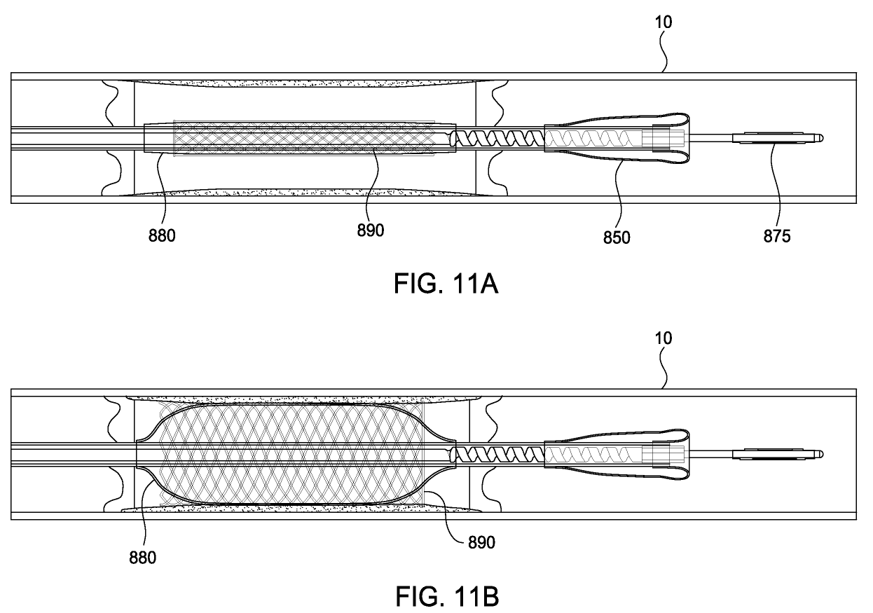

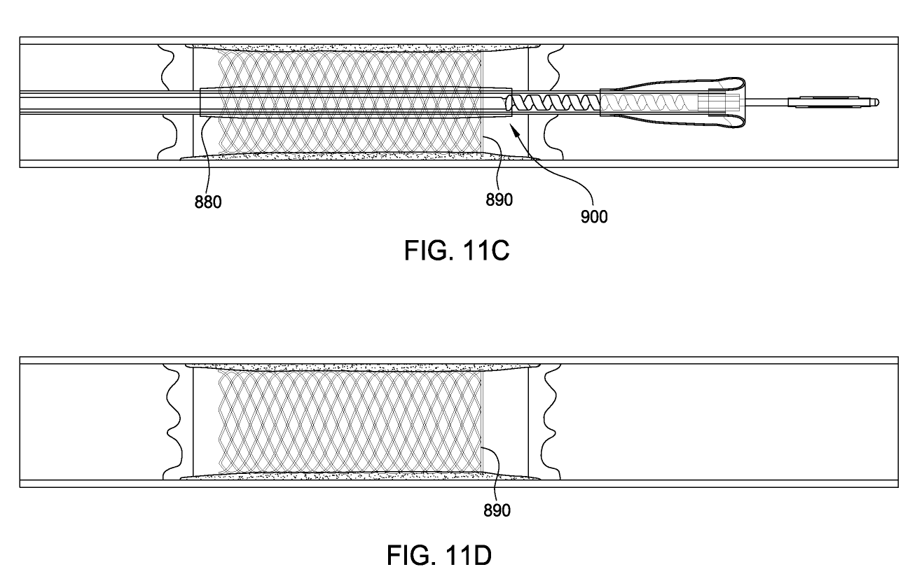

FIGS. 11A-11D show the sequential operation of the surgical device 900. In FIG. 11A, the surgical device 900 has just completed cutting through the occlusion in a manner similar to the process described with respect to FIGS. 9A-9F. After deflating the forwarding balloon 850 and the second balloon 875, the surgical device 900 is positioned in the artery such that the dilatory balloon 880 is located along the previously occluded area. It can be seen that a stent 890 is disposed around the dilatory balloon 880. The stent 890 is shown in the unexpanded position. In one embodiment, the balloon 880 is slightly inflated to provide a gripping pressure against the unexpanded stent 890 to temporarily retain the stent in place during insertion. The length of the stent 890 may be any length suitable to support the expanded lumen. In one embodiment, the length of the stent 890 is at least half the length of the occlusion. Exemplary stents include nitinol stent, polyethylene stents, or any suitable stent known to a person of ordinary skill in the art. In FIG. 11A, a nitinol stent 890 is selected to support the arterial wall. After properly positioning the stent 890, the dilatory balloon 880 is inflated, thereby causing expansion of the stent 890 against the arterial wall, as shown in FIG. 11B. After expansion, the balloon 880 is deflated to disengage from the stent 890, as shown in FIG. 11C. Thereafter, surgical device 900 is removed, leaving behind the expanded stent 890, as shown in FIG. 11D. In another embodiment, a polyethylene stent is disposed on the surgical device. The dilatory balloon is inflated sufficiently to retain the stent for insertion into the artery. The polyethylene stent may include tapered ends to facilitate insertion or removal thereof. After the stent is properly positioned, the balloon may be deflated to disengage from the stent.

While the foregoing is directed to embodiments of the present invention, other and further embodiments of the invention may be devised without departing from the basic scope thereof, and the scope thereof is determined by the claims that follow.

What is claimed is:

1. A tissue removal apparatus, comprising:
a catheter having a lumen;
a rotatable shaft disposed the lumen of the catheter;
a tissue removal portion connected to the rotatable shaft; and
an expandable element circumferentially disposed at a distal end of the catheter, wherein upon expansion, at least a portion of the expandable element extends past the distal end of the catheter.

2. The tissue removal apparatus of claim 1, wherein the rotatable shaft includes an auger portion.

3. The tissue removal apparatus of claim 1, wherein the tissue removal portion comprises one or more blades.

4. The tissue removal apparatus of claim 3, wherein the four blades are used and each of blades are substantially equal in size and length, and one end of each blade is connected to the rotatable shaft.

5. The tissue removal apparatus of claim 3, further comprising a central cutting member.

6. The tissue removal apparatus of claim 1, wherein the tissue removal portion comprises a central cutting member having pointed front end.

7. The tissue removal apparatus of claim 6, wherein the central cutting member includes one or more recesses formed on its outer surface.

8. The tissue removal apparatus of claim 7, wherein a blade is disposable in the one or more recesses.

9. The tissue removal apparatus of claim 1, wherein the tissue removal portion includes a plurality of expandable cutting members.

10. The tissue removal apparatus of claim 9, further comprising a retractable conveying member for expanding the plurality of expandable cutting members.

11. The tissue removal apparatus of claim 1, further comprising a second expandable element.

12. The tissue removal apparatus of claim 11, wherein the second expandable element is conveyed on a tubular.

13. The tissue removal apparatus of claim 12, wherein the tubular is inserted through the rotatable shaft.

14. The tissue removal apparatus of claim 12, wherein the tubular comprises a catheter.

15. The tissue removal apparatus of claim 11, further comprising a third expandable element.

16. The tissue removal apparatus of claim 15, wherein the third expandable element is adapted to deliver a stent.

17. The tissue removal apparatus of claim 1, further comprising a vacuum source connected to the lumen of the catheter.

18. The tissue removal apparatus of claim 1, wherein the catheter includes a plurality of lumens.

19. The tissue removal apparatus of claim 1, wherein the expandable element is adapted to centralize the tissue removal portion in a blood vessel.

20. A method of removing tissue from a blood vessel, comprising:
Inserting into the blood vessel a distal end of a tissue removing apparatus comprised of a catheter having a rotatable shaft disposed therein, a tissue removal portion connected to the rotatable shaft, and an expandable element circumferentially disposed at a distal end of the catheter;
expanding the expandable element against the blood vessel;
extending the tissue removal portion beyond the expandable element; and
rotating the tissue removal portion to remove the tissue, whereby the expandable element prevents the tissue removal portion from perforating the blood vessel.

21. The method of claim 20, wherein expanding the expandable element comprises expanding the expanding element such that a distal end of the expandable element extends beyond a distal end of the catheter.

22. The method of claim 20, wherein the tissue removal portion comprises one or more blades.

23. The method of claim 22, further comprising expanding the one or more blades.

24. The method of claim 20, further comprising retracting a conveying member to expand the one or more blades.

25. The method of claim 20, further comprising delivering a second expandable element from the catheter to the other side of the tissue.

26. The method of claim 20, further comprising installing a stent in the blood vessel after removing the tissue.

27. The method of claim 20, further comprising withdrawal the removed tissue through the catheter.

28. A tissue removal apparatus for removing a tissue in a blood vessel, comprising:
a catheter having at least one lumen;
a conveyor disposed in one of the at least one lumen of the catheter;
a tissue removal portion connected to the shaft; and
an expandable centralizer disposed at a distal end of the catheter, wherein upon expansion, the expandable centralizer is adapted to centralize the tissue removal portion in a blood vessel.

29. The tissue removal apparatus of claim 28, wherein at least a portion of the expandable centralizer extends past the distal end of the catheter after expansion.

30. The tissue removal apparatus of claim 29, wherein the shaft is rotatable.
